(19) United States
(12) Patent Application Publication
(10) Pub. No.: US 2025/0128015 A1
(43) Pub. Date: Apr. 24, 2025

(54) SYSTEM AND METHOD FOR LOW-POWER NEUROMODULATION

(71) Applicants: THE GOVERNING COUNCIL OF THE UNIVERSITY OF TORONTO, Toronto (CA); THE TRUSTEES OF THE UNIVERSITY OF PENNSYLVANIA, Philadelphia, PA (US)

(72) Inventors: Xilin LIU, Toronto (CA); Andrew G. RICHARDSON, Philadelphia, PA (US)

(21) Appl. No.: 18/906,947

(22) Filed: Oct. 4, 2024

Related U.S. Application Data
(60) Provisional application No. 63/591,654, filed on Oct. 19, 2023.

(51) Int. Cl. A61M 21/00

(52) U.S. Cl. CPC A61M 21/00; A61M 2021/0027; A61M 2205/3303; A61M 2210/0662; A61M 2230/10

(57) ABSTRACT

There is provided a low-power neuromodulation system and a method for low-power neuromodulation. The system including a controller to receive electroencephalogram (EEG) signals from one or more pairs of electrodes, the controller including a processor, memory, integrated circuitry, field programmable gate array, or a combination thereof, to execute: an analog front-end (AFE) module to digitize and amplify the received EEG signals; a processing module to classify sleep stages using a deep learning model, the deep learning model taking the digitized and amplified EEG signals as input, the deep learning model including representation learning to capture time-invariant information from the input, sequential learning to capture the sleep stage transition using features encoded in the representation learning, and a dense network to generate a prediction for the sleep stages using the captured time-invariant information and the captured sleep stage transitions; and an output module to output the classification of sleep stages.

### TECHNICAL FIELD

The following relates, generally, to brain signal recordation and modulation; and more particularly, to a system and method for low-power neuromodulation.

### BACKGROUND

Neuromodulation is a type of therapy that involves stimulating specific areas of the nervous system to modify the activity of neurons in the brain to alleviate symptoms of various neurological disorders. One of the most common applications of neuromodulation is in the treatment of sleep disorders. Sleep disorders, such as insomnia and sleep apnea, can lead to a variety of health problems, including cardiovascular disease, diabetes, depression, and dementia including Alzheimer's disease. With the aging of the population, there is a substantial need for highly effective treatments to address sleep disorders and slow down the onset of dementia and Alzheimer's disease.

Sleep is not a homogenous state; it is composed of an alternation of rapid eye movement (REM) sleep and multiple stages of non-REM (NREM) sleep that can be classified according to electrical activity in the brain; i.e., with the use of electroencephalogram (EEG). The high-amplitude, low-frequency (approx. 1 Hz), slow-wave activity (SWA) detected in EEG during the deepest stages of NREM sleep is essential for neurocognitive and many other functions. Disruptions of SWA can lead to deficits in long-term memory formation. Conversely, enhancing SWA during NREM sleep can improve memory. However, conventional sleep studies require large instruments to record signals and data associated with sleep; which are only feasible in sleep labs. The high cost of these studies and the discomfort experienced by users wearing electrodes during sleep substantially prevent better uptake. Furthermore, these limitations in uptake substantial hinder discoveries in the field of neuroscience and sleep.

### SUMMARY

In an aspect, there is provided a low-power neuromodulation system, the system comprising a controller to receive electroencephalogram (EEG) signals from one or more pairs of electrodes, the controller receives power from a power source, the controller comprising a processor, memory, integrated circuitry, field programmable gate array, or a combination thereof, to execute: an analog front-end (AFE) module to digitize and amplify the received EEG signals; a processing module to classify sleep stages using a deep learning model, the deep learning model taking the digitized and amplified EEG signals as input, the deep learning model comprising representation learning to capture time-invariant information from the input, sequential learning to capture the sleep stage transition using features encoded in the representation learning, and a dense network to generate a prediction for the sleep stages using the captured time-invariant information and the captured sleep stage transitions; and an output module to output the classification of sleep stages.

In a particular case of the system, the representation learning comprises one or more Convolutional Neural Network (CNN) paths, each CNN path trained to learn features from the received EEG signals using a distinct time scale.

In another case of the system, the features are in either an analog domain or a digital domain.

In yet another case of the system, the output of the representation learning and the output of the sequential learning are provided as residual connections to the dense network.

In yet another case of the system, the system further comprising an auditory stimulator, and wherein the processing module further determines neuromodulation auditory feedback using the classified sleep stages such that the auditory feedback is delivered in phase with sleep oscillation, and wherein the output module further outputs the auditory feedback with the auditory stimulator.

In yet another case of the system, the auditory feedback comprises in-phase pink noise.

In yet another case of the system, the processing module filters the received EEG signals to determine occurrence of slow-wave oscillation, and wherein the processing module outputs the auditory feedback during specific phases of the occurrence of the slow-wave oscillation.

In yet another case of the system, the received EEG signals comprise only one EEG channel.

In yet another case of the system, the deep learning model uses long kernels using at least one of: memory hierarchies, employing several processing elements (PEs) as part of the processor to manage different segments of the received EEG signals or kernel concurrently, kernel compression, and use of a minimum precision per kernel.

In yet another case of the system, wherein the processing module performing dynamic supply voltage scaling by instructing adjustment of voltage from the power source based on the layers of the deep learning model being processed, instructing lower supply voltage during low-precision layers.

In another case, there is provided a method for low-power neuromodulation, the method comprising: receiving electroencephalogram (EEG) signals; digitizing and amplifying the received EEG signals; classifying sleep stages using a deep learning model, the deep learning model taking the digitized and amplified EEG signals as input, the deep learning model comprising representation learning to capture time-invariant information from the input, sequential learning to capture the sleep stage transition using features encoded in the representation learning, and a dense network to generate a prediction for the sleep stages using the captured time-invariant information and the captured sleep stage transitions; and outputting the classification of sleep stages.

In a particular case of the method, the representation learning comprises two Convolutional Neural Network (CNN) paths, each CNN path trained to learn features from the received EEG signals with different time scales.

In another case of the method, the sequential learning comprises a bidirectional Long Short-Term Memory (LSTM) network.

In yet another case of the method, the output of the representation learning and the output of the sequential learning are provided as residual connections to the dense network.

In yet another case of the method, the method further comprising determining neuromodulation auditory feedback using the classified sleep stages such that the auditory feedback is delivered in phase with sleep oscillation, and the method further comprising outputting the auditory feedback.

In yet another case of the method, the auditory feedback comprises in-phase pink noise.

In yet another case of the method, the method further comprising filtering the received EEG signals to determine occurrence of slow-wave oscillation, and wherein the auditory feedback is outputted during specific phases of the occurrence of the slow-wave oscillation.

In yet another case of the method, the received EEG signals comprise only one EEG channel.

In yet another case of the method, the deep learning model uses long kernels using at least one of: memory hierarchies, employing several processing elements (PEs) as part of the processor to manage different segments of the received EEG signals or kernel concurrently, kernel compression, and use of a minimum precision per kernel.

In yet another case of the method, the method further comprising performing dynamic supply voltage scaling by instructing adjustment of voltage based on the layers of the deep learning model being processed, instructing lower supply voltage during low-precision layers.

These and other aspects are contemplated and described herein. It will be appreciated that the foregoing summary sets out representative aspects of the system and method to assist skilled readers in understanding the following detailed description.

### DESCRIPTION OF THE DRAWINGS

A greater understanding of the embodiments will be had with reference to the Figures, in which:

FIG. 1 illustrates an exemplary high-level block diagram for sleep circuit manipulation and an example application of such embodiment for sleep modulation;

FIG. 2 illustrates a physical example of a 3-D printed flexible in-ear device, in accordance with various embodiments;

FIG. 3 is an example block diagram for producing a digitized signal from an acquired EEG signal, in accordance with various embodiments;

FIG. 4 illustrates a block diagram of an example implementation of a machine-learning model, in accordance with various embodiments;

FIG. 5 shows an example schematic for an auditory stimulator, in accordance with various embodiments;

FIG. 6 shows a block diagram of a system for low-power neuromodulation, according to an embodiment;

FIG. 7 illustrates an exemplary block diagram of the system of FIG. 6 as can be used for sleep modulation;

FIGS. 8A and 8B show an illustrative block diagram of an approach for sleep modulation using the system of FIG. 6;

FIG. 9 is a diagram showing a particular architecture for a hybrid deep learning model, in accordance with various embodiments;

FIG. 10A illustrates a simplified circuit schematic of an example of an analog filter for slow-wave oscillation detection;

FIG. 10B illustrates a simplified circuit schematic of an example of a pink noise generator implemented in the analog domain;

FIG. 11A shows an experimentally measured frequency response of the biquad filter;

FIG. 11B shows an experimentally measured time-domain and spectrum of generated pink noise; and

FIG. 12 a flowchart of a method for low-power neuromodulation, according to an embodiment.

### DETAILED DESCRIPTION

For simplicity and clarity of illustration, where considered appropriate, reference numerals may be repeated among the Figures to indicate corresponding or analogous elements. In addition, numerous specific details are set forth in order to provide a thorough understanding of the embodiments described herein. However, it will be understood by those of ordinary skill in the art that the embodiments described herein may be practised without these specific details. In other instances, well-known methods, procedures and components have not been described in detail so as not to obscure the embodiments described herein. Also, the description is not to be considered as limiting the scope of the embodiments described herein.

Various terms used throughout the present description may be read and understood as follows, unless the context indicates otherwise: “or” as used throughout is inclusive, as though written “and/or”; singular articles and pronouns as used throughout include their plural forms, and vice versa; similarly, gendered pronouns include their counterpart pronouns so that pronouns should not be understood as limiting anything described herein to use, implementation, performance, etc. by a single gender. Further definitions for terms may be set out herein; these may apply to prior and subsequent instances of those terms, as will be understood from a reading of the present description.

Any module, unit, component, server, computer, terminal or device exemplified herein that executes instructions may include or otherwise have access to computer readable media such as storage media, computer storage media, or data storage devices (removable and/or non-removable). Computer storage media may include volatile and non-volatile, removable and non-removable media implemented in any method or technology for storage of information, such as computer readable instructions, data structures, program modules, or other data. Examples of computer storage media include RAM, ROM, EEPROM, flash memory or other memory technology. Any such computer storage media may be part of the device or accessible or connectable thereto. Further, unless the context clearly indicates otherwise, any processor or controller set out herein may be implemented as a singular processor or as a plurality of processors. The plurality of processors may be arrayed or distributed, and any processing function referred to herein may be carried out by one or by a plurality of processors, even though a single processor may be exemplified. Any method, application or module herein described may be implemented using computer readable/executable instructions that may be stored or otherwise held by such computer readable media and executed by the one or more processors.

Various approaches to non-invasive neuromodulation generally utilize either stationary or wearable headsets to record neural signals. Typically, electrodes are positioned on the user's head or behind their ear which communicate with an external computer to perform the neuromodulation algorithms. Such approaches generally have a number of limitations, including that the wearables are cumbersome and uncomfortable for extended periods of wear, and are aesthetically unappealing, which hinders their application. Additionally, the use of external computer-based processing often leads to undesirable latency due to the requirements of wireless data communication. While the use of cable connections is both inconvenient and unsafe for users, especially when the devices are connected to external power and ground.

Further, closed-loop sleep modulation is a paradigm that can be used to treat sleep disorders and enhance sleep benefits. However, for sleep modulation, typically subjects need to be wire-connected to rack-mount instrumentation for data acquisition, which negatively affects sleep quality. Further, conventional real-time sleep stage classification algorithms give limited performance. Particularly, closed-loop sleep approaches are generally only feasible if the sleep stages can be detected accurately in real time and the stimulus signal can be delivered in phase with the sleep oscillation. Data acquisition also needs to minimize adverse effects on the subjects' sleep process to receive accurate data, which generally excludes the use of rack-mounted instrumentation. Advantageously, embodiment of the present disclosure provide a relatively small and non-intrusive system that supports closed-loop on-device sleep modulation, which can be worn comfortably during sleep.

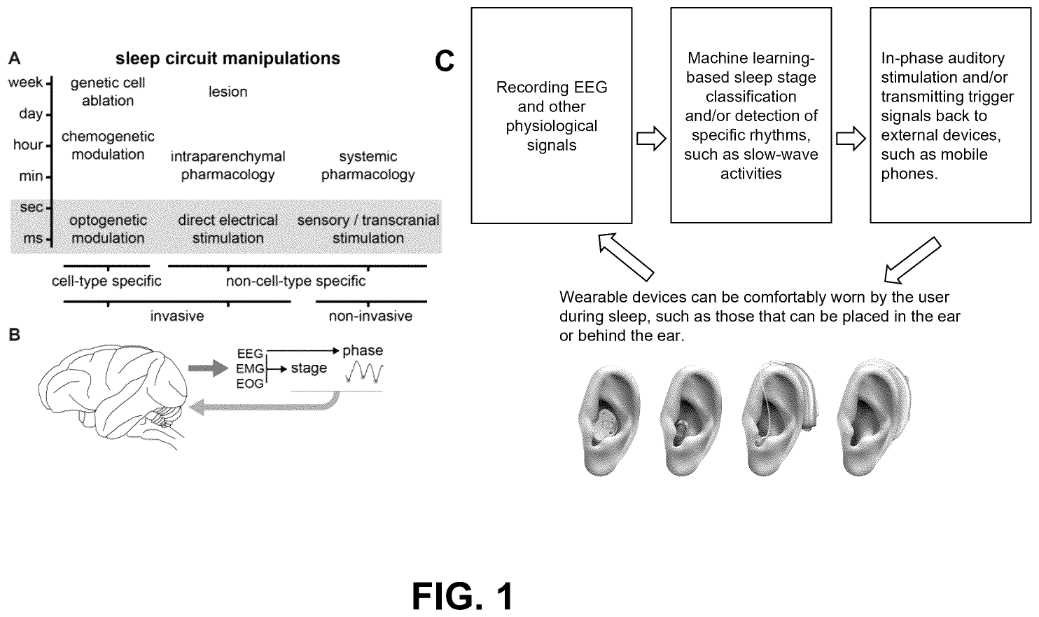

Advantageously, the present inventors have determined that EEG signals collected from the ear canal can be utilized to detect sleep stages. The present inventors have developed the present embodiments, described herein, of ultra-precise, miniature electrodes designed for recording EEG signals in the ear canal. Such dry electrodes that can advantageously be placed inside the ear to enable comfortable wear for extended periods. Further advantageously, in some embodiments of the present invention, a micro-electromechanical-system (MEMS)-based speakers can be used to generate auditory stimulation, such as pink noise or music, to modulate sleep conditions. FIG. 1 illustrates an exemplary high-level block diagram of embodiments of the present invention for sleep circuit manipulation and an example application of such embodiment for sleep modulation.

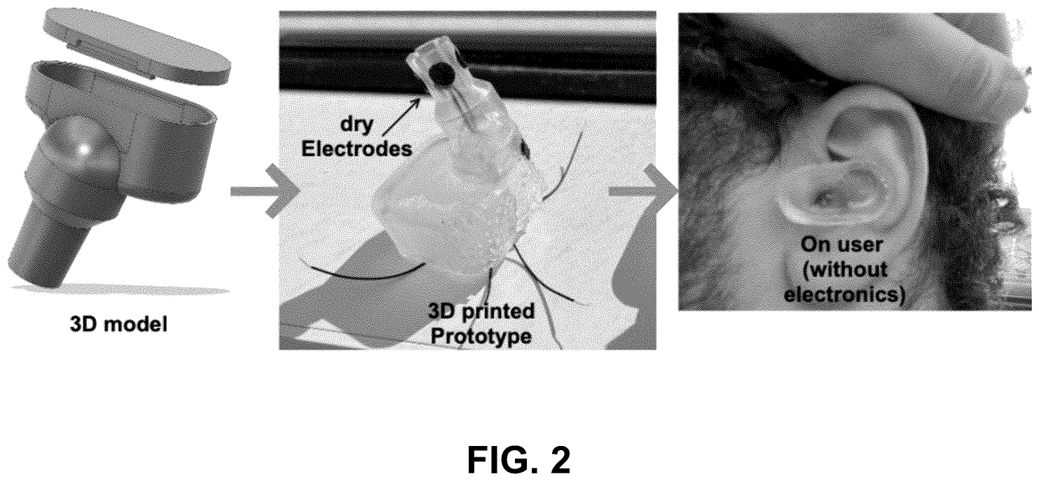

Embodiments of the present disclosure provide in-ear electrodes to record electroencephalogram (EEG) signals from the ear canal that are advantageously dry; i.e., without requiring the use of any gel or paste. In a particular case, MXene, a two-dimensional material composed of transition metal carbides, nitrides, or carbonitrides, has been determined by the present inventors to be able to be used for manufacture of the dry electrodes due to its high conductivity and flexibility. The MXene-based dry electrodes can be produced as small and thin metal layers with a patterned shape that matches the shape of the ear canal. The thin and flexible metal layer conforms to the shape of the ear canal, providing a comfortable fit for the user. The MXene layer is then covered with a thin layer of polymer to insulate the metal from the ear canal and to provide a biocompatible surface. The use of MXene-based dry electrodes offers several advantages over conventional wet electrodes. They are easy to apply, do not require gel or paste, and do not cause skin irritation or discomfort. Additionally, they are generally more hygienic and reusable compared to conventional wet electrodes. FIG. 2 illustrates a physical example of the 3-D printed flexible in-ear device with MXene-based dry electrodes.

Embodiments of the present disclosure further provide integrated circuits (ICs) for EEG recording with multiple feedback loops and chopping techniques to reduce noise and improve signal quality. The ICs can be fabricated using, for example, complementary metal-oxide-semiconductor (CMOS) technology. The present inventors have developed ICs that include multiple feedback loops to provide high gain and low noise amplification of the EEG signals. The feedback loops also help to maintain the stability of the amplifier, improving the accuracy of the recorded signals. In addition, the ICs can, in some cases, incorporate chopping techniques to reduce the effects of low-frequency noise and DC offset; which can interfere with the EEG signals. The chopping technique can include alternating the polarity of the input signal; which effectively cancels out low-frequency noise. The feedback loops can also be used to operate in a chopped mode, further reducing noise and improving signal quality. Advantageously, the ICs for EEG recording with multiple feedback loops and chopping techniques can provide high-quality EEG signals with minimal noise and distortion. This high-quality performance is critical for recoding EEG signals from the ear canal with sufficient details for the detection of brain states. An example input referred noise of such EEG recording front-end is around 0.5 μV to 10 μV.

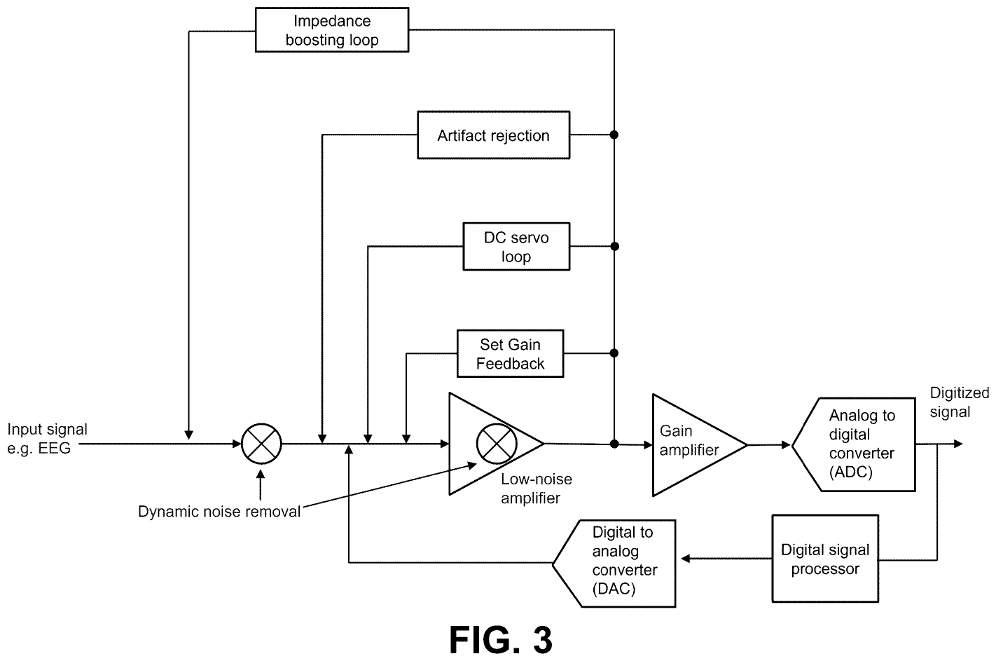

FIG. 3 shows an example block diagram for producing a digitized signal from an acquired EEG signal. In the illustrated technique, dynamic noise removal techniques, such as chopping or auto-zeroing, may be used to reduce noise. Various feedback loops can be used to enhance input impedance, remove artifacts, remove DC offset, and set the gain. An amplifier may be used to provide additional gain and drive the ADC that follows. The ADC can be used to digitize the amplified signal, and it may be shared across multiple analog channels. A digitally assisted loop may also be utilized for artifact, offset and/or noise cancellation. In an example, the low-noise amplifier input referred noise can be around 0.5 μV to 10 μV and the ADC resolution can range from 12 bits to 24 bits.

In some cases, a DC servo loop can be used to eliminate any large offset that appears at the electrode interface, as exemplified in FIG. 3. In addition, a feedback loop can be used to increase the input impedance of the front-end, which can be beneficial because dry electrodes used for in-ear recordings typically have high impedance. A high input impedance in the analog front-end is useful to minimize signal attenuation and distortion. In addition, a feedback loop can be used to eliminate motion-related artifacts in the recording. The feedback loop may be used to detect a common-mode signal from the output and cancel it from the input. An additional amplifier may be used after the low-noise amplifier to provide extra gain and sufficient driving capacity for the subsequent stages, particularly the analog-to-digital converter (ADC). The ADC may be shared across multiple input channels and may utilize oversampling to enhance noise performance. An ADC resolution typically ranges from 12 to 24 bits, producing digitized EEG signals as the output. The digitized signals can be processed by a digital signal processor (DSP), and the output may be used to cancel offset, artifacts, or noise. In some cases, such cancellation can use a digital-to-analog converter (DAC).

Embodiments of the present disclosure also provide a machine learning approach for the detection of various brain states, such as sleep stages. In some cases, a two-stage model can be performed on-chip. In a first stage, feature extraction can be performed. This can be achieved, for example, by determining various statistical features, such as the power spectral density, bispectral coherence, and higher-order spectra of the EEG signal. These features can then be pre-processed and fed into a second stage. The second stage can include classification, which involves using a trained algorithm to classify the sleep stage based on the extracted features. The algorithm can be optimized to operate on low-power hardware to support real-time inference on the device. Such approach can provide various advantages, for example, low latency, safety without cybersecurity concerns, and robustness with low power consumption, as there is no reliance on external devices. The machine learning model can be trained using a large dataset of annotated EEG signals collected during sleep studies. The dataset can be pre-processed and labeled with the corresponding sleep stage, for example, wakefulness, light sleep, deep sleep, and REM sleep. The model can be trained using, for example, deep learning techniques to achieve high accuracy in sleep stage classification; such as using convolutional neural networks, recurrent neural networks, transformers, or a hybrid combination of them.

In a particular case, the machine learning models can be trained to sense oscillatory activity specific for certain brain states after receipt of the captured ear EEG signals; which can be used to entrain the oscillations with auditory stimuli to prolong or enhance these brain states. For example, slow-wave sleep can be prolonged, and memory enhanced, with closed-loop auditory stimulation during sleep.

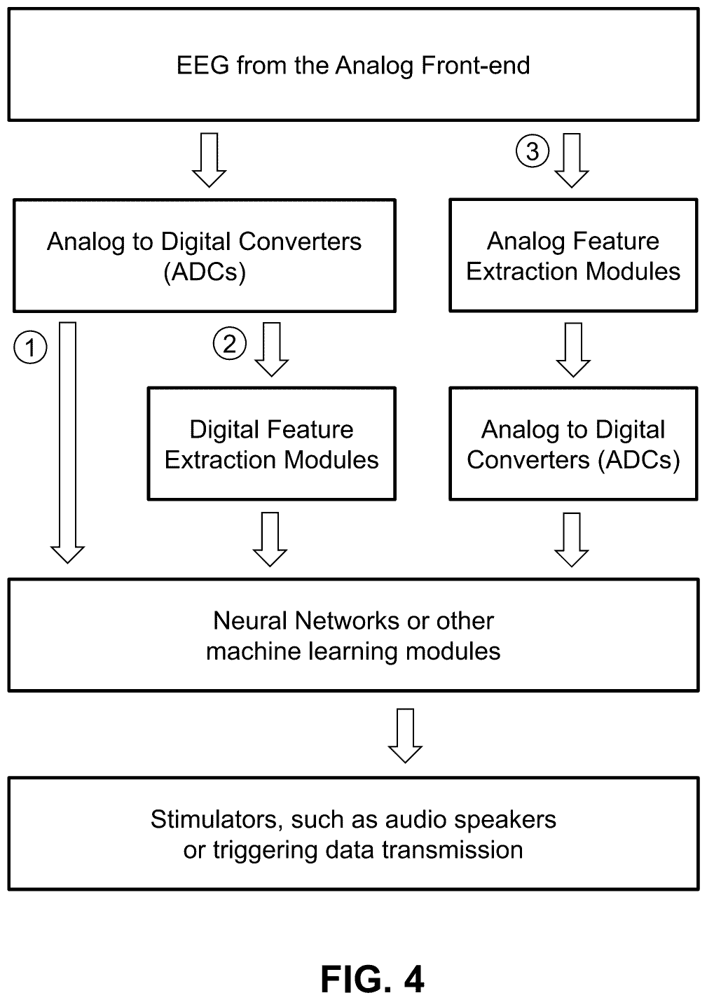

FIG. 4 illustrates a block diagram of an example implementation of the machine-learning model. The machine learning model of this example can follow several pathways. First, the amplified signal may be digitized and directly input into the neural network. Second, features may be selected and extracted from the digitized EEG signal before being fed into the neural network. Third, features may be selected and extracted in the analog domain, then digitized and provided to the machine learning model. Variations arising from analog feature extraction can be compensated by retraining the neural network model to account for any errors. The machine learning model could be a neural network, CNN, LSTM, transformer, or a combination of these architectures. The output of the model is the identified sleep stage, which can be used to trigger stimuli, such as auditory stimulation, in conjunction with phase information extracted from slow-wave signals. Additionally, the classification results may be used to initiate data transmission, such as communicating the output to other devices, like mobile phones.

As illustrated in the example of FIG. 4, there can be multiple signal paths. A first signal path, marked as ‘\textcircled{1}’, has a digitized time-domain signal that can be directly input into machine learning modules, such as neural networks, CNNs, LSTMs, transformers, or others. A second signal path, marked as ‘\textcircled{2}’, has features that may be selected and pre-extracted before being fed into the machine learning modules. A third signal path, marked as ‘\textcircled{3}’, has features that can also be extracted in the analog domain prior to digitization. Variations arising from analog feature extraction can be compensated by retraining the neural network model to account for any errors. In some cases, ADCs may be reused.

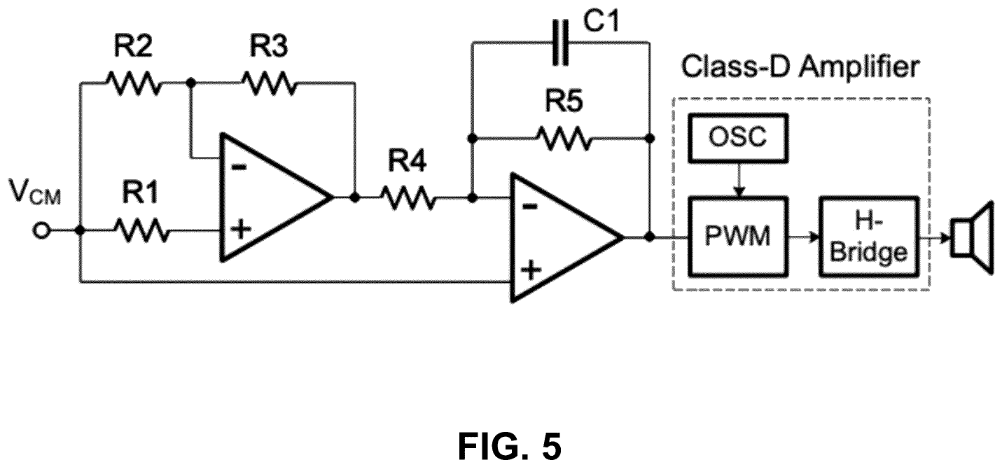

In embodiments of the present disclosure, in order to deliver neuromodulation, an auditory stimulator can be provided. The auditory stimulator for sleep modulation can include, for example, a MEMS (micro-electromechanical systems) speaker and a class-D amplifier. The MEMS speaker can be used to generate music or in-phase pink noise as a form of stimulation. The class-D amplifier can be used to convert digital signals to analog signals using pulse-width modulation. Adventurously, the auditory stimulator is small and lightweight, making it easy to integrate into the in-ear device. In some cases, the auditory stimulator can be controlled by an external microcontroller, which can send the desired stimulation signal to the device. FIG. 5 shows an example schematic for the auditory stimulator.

Further, advantageously, embodiments of the present discourse can be used for sleep modulation using closed-loop operations performed solely on the device. In some cases, sleep stage classification can be performed using a lightweight deep learning (DL) model accelerated by a low-power field-programmable gate array (FPGA) device or an application specific integrated circuits (ASIC) device. The DL model can use a single channel or multiple channels of electroencephalogram (EEG) as input. One or more convolutional neural networks (CNNs) can be used to capture general and detailed features, and a long-short-term memory (LSTM) network can be used to capture time-variant sequence features. In some cases, an 8-bit quantization can be used to reduce the computational cost without compromising performance. The present inventors have validated the DL model using a public sleep database containing 81 subjects, achieving a classification accuracy of 85.8% and a F1-score of 79%. The DL model has also shown the ability to be generalized to different channels and input data lengths.

Advantageously, embodiments of the present discourse provide dynamic quantization for machine learning inference. Quantization is often employed in machine learning inference to lower the computational demands compared to using floating-point representations. Traditional quantization, however, is uniform and generally does not effectively balance the trade-off between accuracy and energy efficiency. In contrast, the present embodiments leverage the multiplier's feature that facilitates energy-efficient computations across various bit widths. A dynamic quantization technique is provided that permits the utilization of different levels of quantization for separate layers. Additionally, a dynamic mapping scheme between the layers is provided to enable maximization of the utilization of the dynamic range within the constraints of a limited word length.

Advantageously, embodiments of the present discourse also provide dynamic supply voltage scaling. Dynamic supply voltage scaling is employed to minimize the power consumption of digital circuits. In embodiments of the present discourse, the voltage can be adjusted based on the layers being processed; where lower supply voltage is used for low-precision parts, reducing power. Additionally, in some cases, error monitoring is performed. If a logic error is detected, the supply voltage can be increased to rectify it. This tailored voltage scaling not only conserves energy but also maintains accuracy through real-time error detection and correction.

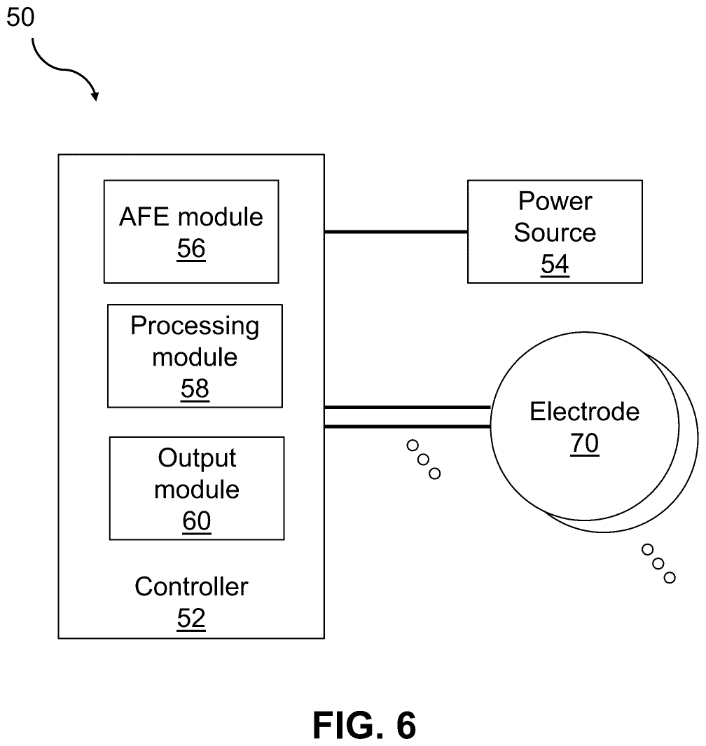

Turning to FIG. 6, a conceptual diagram of a system for low-power neuromodulation 50, according to an embodiment, is shown. The system 50 comprises a controller 52, a power source 54 (e.g., batteries), and two or more in-ear electrodes 70. The two or more electrodes 70 are electrically connected to the controller 52, which supplies an electrical current through the electrodes 70 via the power supplied by the power source 54. In some cases, the system 50 can also include an auditory stimulator connected to the controller 52 and receive power via the power source 54. In some cases, the controller 52 can include a field-programmable gate array (FPGA), one or more processors, or other circuitry. In some cases, the controller 52 can include or be in communication with one or more memory units, such as flash memory. In further cases, the controller 52 can be implemented using dedicated circuitry. The controller 52 can also include an interface to interface with the user via input devices/elements, and/or output devices/elements, and/or interface with other computing devices. The system 50 can include circuitry for power management located associated with the power source 54 or the controller 52. The system 50 can be located in a housing for insertion into a user's ear; such as the form of the example 3D-printed prototype illustrated in FIG. 2.

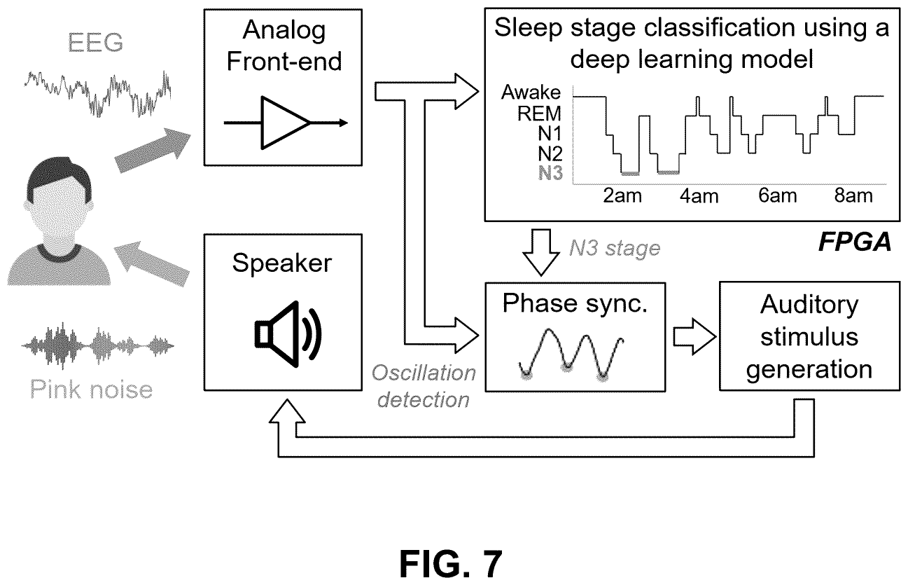

Advantageously, the system 50 can provide closed-loop sleep modulation in a way that is self-contained and potentially miniaturized. FIG. 7 illustrates an exemplary block diagram of the system 50 as can be used for sleep modulation. In this example, the system 50 uses a single-channel EEG as input. Sleep stage classification is performed using a light-weight deep learning (DL) model implemented in a low-power field-programmable gate array (FPGA). Auditory stimulation is activated on the basis of the specific sleep stage and the detected sleep oscillation.

Other approaches for developing a machine learning model to classify sleep stages generally have a number of significant limitations. For example, such approaches are generally demand too much computational resources that are not available in energy-constrained wearable devices; use too many input channels that result in high power dissipation for signal acquisition and causing inconvenience in electrode placement; and use long time series as input (for example, more than a minute), which causes latency in real-time operation and is not suitable for closed-loop modulation. In contrast, the system 50 can execute a DL model that uses only one EEG channel as input with a segment of 20 or 30 seconds. A sliding window with overlap can be used in some cases to further reduce inference latency. In an example experiment conducted by the present inventors, a DL model consisted of only 1.28 million parameters, which makes it suitable for, for example, FPGA, or ASIC implementation.

Generally, for machine learning inference, a significant power cost is the memory movement between memory (e.g., SRAM) and the processing unit. Conventional machine learning accelerator designs generally focus on optimizing processing unit designs for small kernels (for example, as commonly used for computer vision applications). In contrast, the present embodiments provide a dedicated controller 52 that minimizes data movement between memory and processing units. This is particularly significant during machine learning inference, where generally the greatest power consumption comes from moving data between memory and processing units. Unlike other machine learning accelerator designs that primarily target small kernels (such as those used in computer vision applications), the system 50 is tailored to neurological signals (i.e., EEG) that typically require a long kernel (i.e., large filter dimension) to capture low-frequency time-domain signals.

Long kernel sizes can be utilized in machine learning models to detect neurological anomalies from EEG readings due to several factors. For example, firstly, EEG data reflects time-based brain activities, and a majority of neurological disorders present themselves as distinct temporal anomalies or patterns within these readings. Lengthy kernels in CNNs can be used to effectively process and comprehend extensive temporal segments of EEG data; facilitating the identification of prolonged patterns or distinctive features. Second, these elongated kernels are adept at isolating lower frequency components, which are useful for EEG studies because numerous neurological ailments correlate with discrepancies in specific frequency ranges. Third, the layered architecture of CNNs, when integrated with extensive kernels, fosters a tiered feature extraction process. In relation to EEG data, this suggests that CNNs can discern both rudimentary (like spikes or waveforms) and intricate (like oscillatory sequences or bursts) features that can be very useful for diagnosing neurological conditions. Lastly, EEG readings frequently contain extraneous disturbances such as muscle movements, ocular activities, or outside electrical disruptions. Long kernel sizes assist in separating genuine neurological attributes from these fleeting disturbances, enhancing the accuracy of the analysis.

In particular embodiments, the system 50 can use the following approaches to optimize handling of long kernels:
Memory hierarchies: the system 50 can use a large on-chip SRAM to store the kernels, ensuring quick access times and allowing reduction across multiple CNN operations. Meanwhile, the system 50 can use burst reads to fetch data from off-chip DRAM.
Parallelism: the system 50 can deploy several processing elements (PEs) to manage various segments of the input data or kernel concurrently, enhancing computation speed. Additionally, the system 50 can utilize pipelining to simultaneously compute different sections of the kernel or data over time.
Kernel compression: the system 50 can compress the kernel without loss of information. Once loaded onto the accelerator, the kernels can be decompressed for computation. This saves memory bandwidth and storage.
Flexible precision: the system 50 can use a minimum precision per kernel to reduce hardware overhead without degradation in performance.

Additionally, the system 50 allows can use an energy-efficient multiply-accumulate operation (MAC) macro that can supports various bit-width. MAC operations are generally performed repeatedly in the inference of ML models. Traditional approaches to speed up these operations rely on using a fixed bit width, such as 8 or 16 bits. In contrast, the system 50 advantageously uses a MAC macro that supports energy-efficient computation across various bit widths; made possible by using analog and mixed-signal design.

The MAC operation generally consists of multiplying two numbers, A and B, and then adding the result to an accumulator. A particular challenge is that digital multiplication, especially at high resolutions, can be power-intensive. Consequently, optimizing the power usage of the MAC operation can be very useful to minimize the energy footprint of machine learning inference. Certain approaches to overcome this problem requires adhering to a fixed bit width, commonly settling on 8 or 16 bits. Distinctively, the system 50 can use a MAC with adaptable bit width, allowing A and B to differ in their resolutions. This adaptability prevents undue power consumption associated with high-resolution multiplications when, at times, resolutions as low as 4 bits might suffice, despite many standard multipliers supporting 16 bits or more.

For an FPGA implementation, the MAC can remain fundamentally digital. However, for an application-specific integrated circuit (ASIC) implementation, the system 50 can use analog computation. For example, a digital-to-analog converter (DAC) can serve as an analog platform for multiplication. In this case, the number A is represented in the DAC's code, while B is mirrored as a reference current; resulting in the DAC's output effectively being the product of A and B. This approach allows for compactness, speed, and efficiency, especially at bit widths below 8. In some cases, the system 50 can use multiple processing elements which is beneficial for concurrent multiplications, thereby enhancing throughput and significantly limiting the transfer of coefficients.

The controller 52 can execute code on the processor and/or integrated circuitry to implement the functions of a number of conceptual modules, including: an analog front-end (AFE) module 56 for EEG acquisition in order to detect sleep oscillation; a processing module 58 for sleep stage classification and closed-loop control; and an output module 60 for outputting the classification and/or delivering auditory feedback.

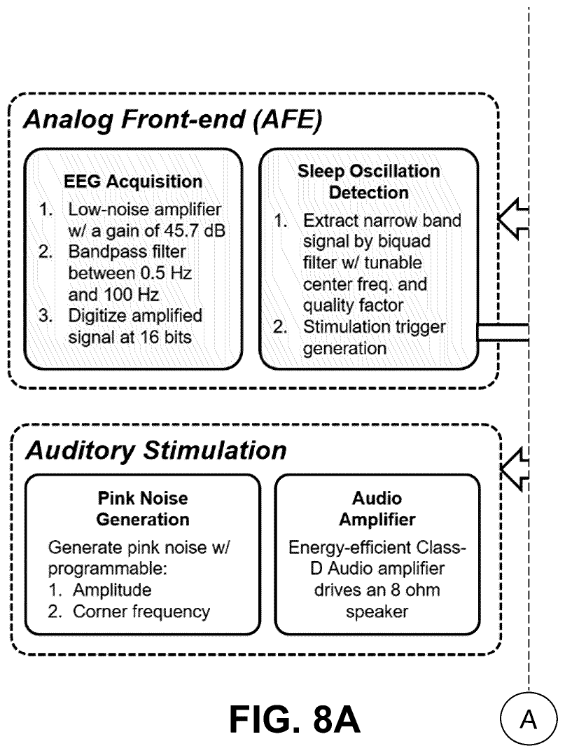

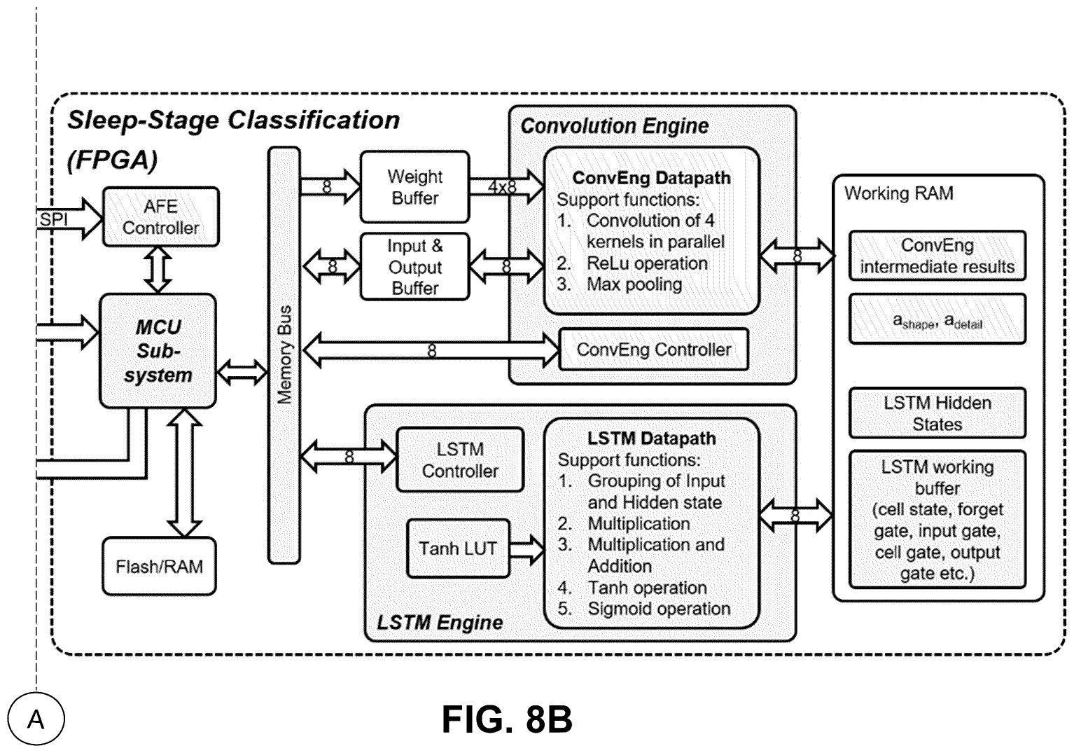

FIGS. 8A and 8B show an illustrative block diagram of an exemplary approach for sleep modulation using the system 50 and also illustrating some example functions of the conceptual modules. In this example, the controller 52 is implemented on an FPGA. A microcontroller unit (MCU) block can be integrated in the FPGA to manage system control. The weights of the DL model can be stored in the memory units (e.g., flash memory) and loaded to the data engine for processing. The MCU monitors the status of the data engine and loads the corresponding weights into the buffers.

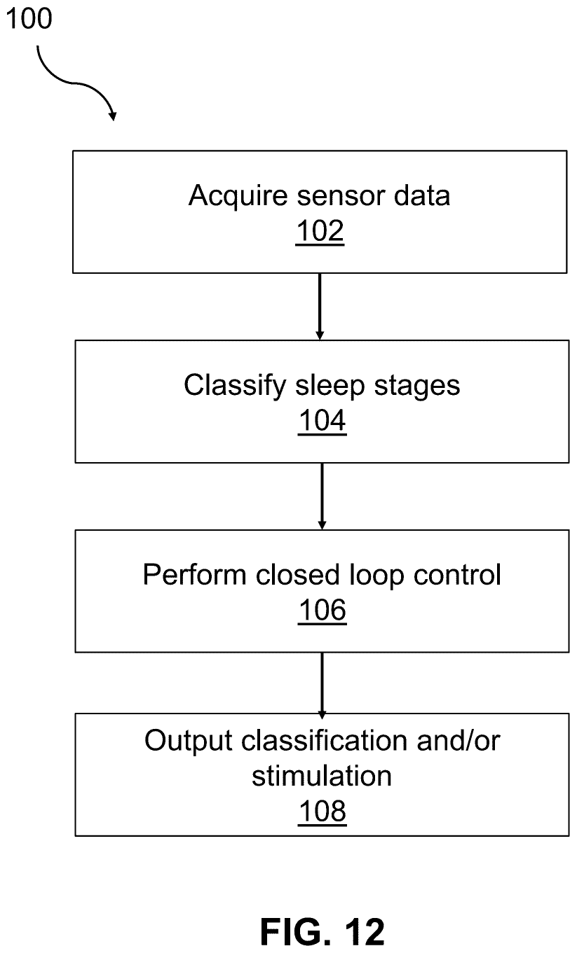

Turning to FIG. 12, a flowchart for a method for low-power neuromodulation 100, according to an embodiment, is shown.

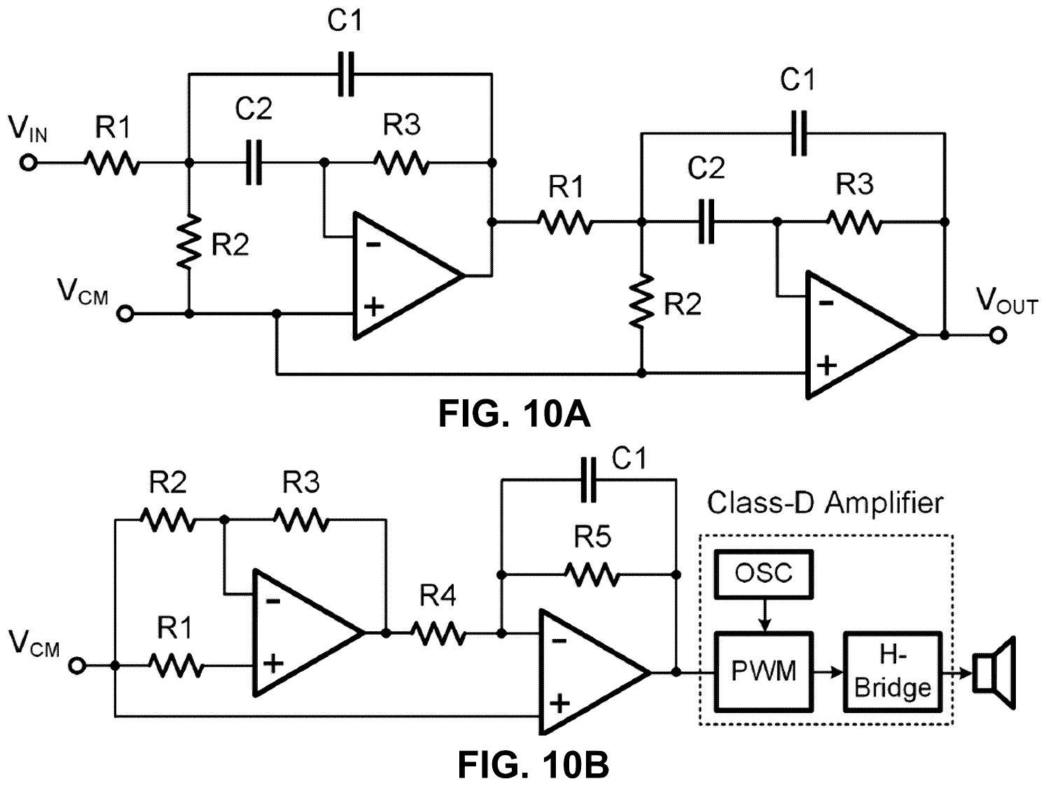

At block 102 of the method 100, the AFE module 56 acquires sensor data from input signals received from the one or more electrodes 70. In some cases, the AFE module 56 can consist of an EEG acquisition path and a sleep oscillation detection path. In a particular case, the EEG acquisition path can use neural amplifier for signal amplification and digitization. In an example, the amplifier can have a gain of 49.5 dB and a digitization resolution of 16 bits. In a particular case, the sleep oscillation detection path can use a 4th-order biquad filter, for example as illustrated in FIG. 10A. FIG. 10A illustrates a simplified circuit schematic of an example of one stage of the biquad filter for slow-wave oscillation detection.

Slow-wave oscillation (SWO) is a specific kind of neural oscillatory activity that is most prominent during deep sleep. SWO is an essential component of sleep architecture and is often considered a marker of sleep quality. SWO reflects synchronized activity in the neural network and plays a key role in the consolidation of memories. The amount and amplitude of SWO tends to decrease with age, which is one of the reasons why older adults often experience less sleep than younger individuals.

SWO can be identified using a narrow band filter set to isolate SWO within a designated frequency band. In FIGS. 5 and 10B, V_{CM} can represent a DC reference voltage. After the signal acquisition, SWO detection can operate concurrently with the machine learning sleep stage identification, as described herein. In a particular case, the phase of the SWO can be used to determine when the pink noise is delivered.

An example circuit for the EEG acquisition path can use only one operational amplifier per biquad core to save power consumption. The transfer function of the biquad filter can given by:

$$H ( s ) = \frac{- s ( \frac{α}{{C}_{1} {R}_{eq}} )}{{s}^{2} + s ( \frac{1}{{C}_{1}} + \frac{1}{{C}_{2}} ) \frac{1}{{R}_{3}} + \frac{1}{{C}_{1} {C}_{2} {R}_{3} {R}_{eq}}}$$

where R_{eq}=(1/R₁+1/R₂)⁻¹ and α=R₄/R₁. The center frequency is:

$${ω}_{0} = 1 / \sqrt{{C}_{1} {C}_{2} {R}_{3} {R}_{eq}}$$

and the quality factor is given by:

$$Q = {[ \frac{\sqrt{{C}_{1} {C}_{2} {R}_{3} {R}_{eq}}}{{R}_{3}} ( \frac{1}{{C}_{1}} + \frac{1}{{C}_{2}} ) ]}^{- 1}$$

A comparator can be used to detect the zero-crossing point of the filtered oscillation signal. The detection signal is provided to the processing module 58 (e.g., the FPGA or ASIC) and a programmable delay can be added before triggering the stimulation.

At block 104 of the method 100, the processing module 58 classifies the sleep stages using the digitized and amplified EEG signals and the detected oscillations. At block 106 of the method 100, the processing module 58 performs closed-loop control for the neuromodulation based using the classified sleep stages to deliver the auditory feedback/stimulation in phase with the sleep oscillation. In some cases, for the closed-loop control, the processing module 58 can have a predetermined delay prior to delivering the auditory stimulation.

In a non-limiting example, the closed-loop control can include:
(1) A polysomnography (PSG) signal is acquired and conditioned, such as through filtering;
(2) Machine learning is performed to identify the sleep stages;
(3) Concurrently with (2), a phase of the slow-wave activity is determined using a suitable technique, such as with a Hilbert transform;
(4) Pink noise stimulation is delivered, dependant on the identified sleep stage and the phase of the slow-wave activity. For example, the system 50 can deliver pink noise at a peak of the slow-wave activity during sleep stage N3.

While machine learning models have been used elsewhere to classify sleep stages, generally such models reply upon manually determined features, including features in the frequency domain (e.g., fast Fourier transform), the time domain (e.g., change in slope sign, waveform length), or the time-frequency domain (e.g., discrete wavelet transform). While such models are generally able to automate sleep scoring, they often render poor generality when applied to different subjects and electrode placements. Other models, without manually determined features, can be used to classify sleep stages; such as deep belief networks and convolutional neural network (CNN) models using time-domain signals directly as input. These models can be used to extract time-invariant features, however, importantly, generally miss time-variant features. To capture time-variant features, such as sleep stage transitions, other models, such as recurrent neural networks (RNNs), can be used.

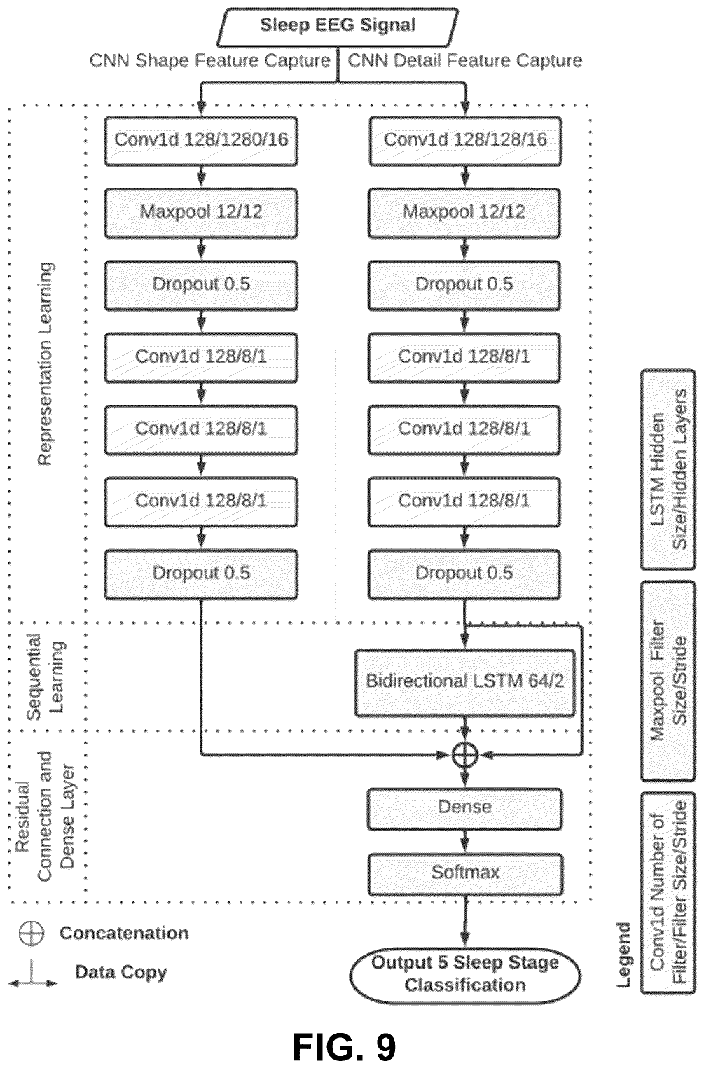

In a particular case of the system 50, the processing module 58 can use a hybrid DL model, for example using both CNN and RNN models. FIG. 9 shows a particular architecture for such hybrid DL model. The following will describe the particular architecture illustrated in FIG. 9; however, it is understood that any suitable ML architecture can be used in the present embodiments. This particular architecture consists of three parts: (1) a first part that uses representation learning to capture time-invariant information from the input vector; (2) a second part that uses sequential learning to capture the sleep stage transition using features encoded in the first part; and (3) a third part that consists of a dense network with a residual connection to generate the prediction.

This hybrid DL model advantageously allows for parallel structure and processing. By integrating two parallel CNN models, neural features can be captured at different frequencies and detail levels. This dual approach enables the model to better analyze neuromodulation applications, enhancing sensitivity to diverse neural patterns, and improve accuracy in predicting or controlling neurological functions. In some cases, the two models can be processed in an application-specific integrated circuit (ASIC) to minimize inference delay, which may be beneficial for closed-loop real-time operation.

In a particular case, the representation learning can consist of two CNN paths; which can be trained to learn features with different time scales. One path can have a large filter size to capture general shape characteristics (αₛₕₐₚₑ) with low-frequency content, and the other path can have a small filter size to capture detail shape characteristics (α_{detail}) with high-frequency content. Both CNN paths can consist of four 1-D convolutional layers, two dropout layers, and one max-pooling layer. Each 1-D convolutional layer can be followed by batch normalization and a rectified linear unit (ReLU) activation function. Two dropout layers can be added to reduce overfitting. To demonstrate the generalizability of such an architecture, the present inventors designed the representation learning to accommodate input EEG signals with a length of 20-seconds or 30-seconds; which can be made possible by adjusting the max-pooling layer and the dropout layers in the two CNN paths, providing a similar output data length for the sequential learning.

Transitions between sleep stages often occur in patterns. In this way, in a particular case, the sequential learning can use a bidirectional LSTM network to capture the sleep transition. α_{detail} can be used as the sole input to the LSTM model for sequential learning, instead of concatenating α_{detail} and αₛₕₐₚₑ. This allows for obtainment of optimal performance at a low computational cost. The sequential learning can output a final forward hidden state h_{f} and a first reverse hidden state hᵣ of the extracted features.

In a particular case, a dense network can generate a final prediction. Both the outputs of the representational learning (αₛₕₐₚₑ, α_{detail}) and the sequential learning (h_{f}, hᵣ) can be taken as input. αₛₕₐₚₑ and α_{detail} can be provided to the dense network as residual connections because they add frequency content that is degraded in sequential learning. h_{f} and hᵣ can be concatenated to provide sufficient time domain features in both the forward and reverse directions.

The convolution layers of the ML model can be used by the processing module 58 to process the input EEG data received from the AFE module 56 and generates α_{detail} and αₛₕₐₚₑ as outputs. Subsequently, the LSTM layers can be used to determine h_{f} and hᵣ from α_{detail}. The convolution layers can then perform the dense operation with h_{f} and hᵣ and output the result (in some cases, through an output buffer). In some cases, the processing module 58 can perform softmax to obtain a final output. In a particular case, to minimize memory access, the convolution layers can processes 4 kernels in parallel, with ReLu and max pooling operations. An address generator in the controller 52 can enable flexible data arrangement of input and output data, so that no additional data moving or reordering is required.

The LSTM layers concatenate the input and hidden states of each layer, so that the convolution layers are used to generate intermediate results of forget gates, input gates, cell gates and output gates. Then the LSTM data path can perform Sigmoid and Tanh operations, as well as other multiplications and additions, to generate an updated cell state and hidden state. In some cases, an optimized interpolation algorithm with a 38-enity Tanh lookup table is used to implement Tanh and sigmoid operations. The interpolation algorithm can share the same multiplier with other LSTM operations for lower hardware complexity.

Advantageously, the deep learning (DL) model architecture of some of the present embodiments integrate two distinct Convolutional Neural Network (CNN) banks; in order to glean both frequency and time domain characteristics inherent in neurological markers present in EEG signals. The first CNN bank can be used to focus on intricate frequency patterns, capturing unique spectral signatures indicative of various neurological states. The second CNN bank can be used to extract temporal sequences, identifying patterns that evolve over time. Further, augmenting this approach, the Long Short-Term Memory (LSTM) layers can be used to recognize and store long-term sequential dependencies within the time domain of the EEG signals. This combination of dual CNN banks and LSTM provides a comprehensive understanding of the EEG data. Notably, the approach of the present embodiments not only elevates the accuracy of neurological disorder detection but achieves this enhanced accuracy while maintaining low computational overhead, minimal memory utilization, and reduced power consumption.

Advantageously, by virtue of the dual Convolutional Neural Network (CNN) banks, the system 50 is inherently modular and ideal for hardware deployments in FPGA or ASIC. FPGA and ASIC generally have the ability to handle parallel computations, making them a suited for the dual CNN banks arrangement; which work to simultaneously extract both frequency and time domain features from EEG signals. This parallelism provides swift and simultaneous data processing, maximizing the hardware's computational capacity. Additionally, the use of LSTM layers can make effective use of the hardware's capabilities. The sequential processing innate to LSTMs can be efficiently pipelined on the hardware, thus making full use of its resources. Moreover, the emphasis on high accuracy with reduced computational overhead means the architecture is streamlined and compact. This compactness can ensure that the architecture can accommodate the hardware's resource constraints while minimizing the need for power-intensive off-chip communications.

The dual CNN banks and LSTM architecture inherently promotes power efficiency. By utilizing distinct CNN banks tailored for precise feature extraction, the model ensures that only relevant components of the EEG signal are processed, thereby eliminating extraneous and power-draining computations. Due to the nature of LSTM layers, which adapt their computations based on the incoming data's significance, means they can dynamically adjust their activity. Not every neuron in the LSTM layer may be consistently active; i.e., during instances where the data sequences are deemed less critical, the LSTM might engage in fewer computations, conserving energy. Furthermore, the focus on minimizing memory overhead implies a design optimized for efficient data storage and access. Given that memory operations, especially those involving off-chip memory, are among the most power-consuming activities in hardware, a model architecture's ability to optimize its data flow and storage can substantially reduce power usage; meaning the model architecture is suitable for low-power applications.

While the dual CNN model and LSTM architecture is described, it is understood that any suitable machine learning architecture for closed-loop neuromodulation can be used. For example, models that encompass linear and logistic regression, support vector machines (SVM), decision trees and random forests, Kalman filters and extended Kalman filters, recurrent neural networks (RNN), transformers, reinforcement learning, and feature extraction approaches like Principal Component Analysis (PCA) or Independent Component Analysis (ICA).

For simplicity of the memory system and better performance, memory blocks in the memory units can use simple dual-port memory. In example experiments, it was determined that the controller 52 can process 20-second input data within 1-second when running at 20 MHz clock. This provides flexibility to support multiple EEG inputs. To further reduce hardware cost, static quantization can be applied for both weight and activation to signed 8 bit. The example experiments bench-marked three calibration approaches: MinMax, entropy, and percentile. Appropriate data shifting and saturation operations were performed in the operations. TABLE 1 summarizes the final resources used for the FPGA implementation in the example experiments.

TABLE 1

| | | Multiplication | Memory |
|---|---|---|---|
| | Parameters | Operation | Required |
| CNN-shape | 425984 | 55836672 | 7936 |
| CNN-detail | 278528 | 9830400 | 7936 |
| LSTM | 196608 | 2960640 | 1920 |
| Residual & | 376832 | 376832 | — |
| dense | | | |
| Total | 1277952 | 69004544 | 17792 |

The pink noise generation was implemented in the analog domain. A 150 kΩ resistor is used as the source of white noise, which is amplified and filtered by a first-order low-pass filter to generate pink noise. The frequency characteristics can be further shaped by the filter. An energy-efficient class-D amplifier was used to drive an 8 Ω piezo transducer speaker.

Auditory stimuli, such as pink noise bursts, provide an effective non-invasive approach to modulate neural activity. Noise bursts evoke temporally precise neural activity along the auditory pathway and interconnected brain networks. When these stimuli are delivered rhythmically, corresponding rhythmic neural activity is produced. When these rhythmic stimuli are timed to occur in-phase with an ongoing brain rhythm, as in the closed-loop approach of the present embodiments, a resonant neural response can occur; boosting the power and/or duration of the brain rhythm. Since brain rhythms in specific stages of sleep have been linked to important functions, e.g., slow oscillations (<1 Hz) in stage N3 are critical for memory consolidation, this closed-loop in-phase approach can provide a neuromodulation approach to enhance the effects of sleep.

At block 108 of the method 100, the output module 60 for outputs the classification and/or outputs auditory stimulation. The classification can be outputted to, for example, a user interface device or another computing device via a suitable communication channel.

FIG. 10B illustrates a simplified circuit schematic of an example of a pink noise generator implemented in the analog domain by the output module 60. In this example, a 150 kΩ resistor is used as a source of the white noise, which is amplified and filtered by a first-order low-pass filter to generate the pink noise. The frequency characteristics can be further shaped by a filter. In an example, an energy-efficient class-D amplifier was used to drive an 8 Ω piezo transducer speaker.

Advantageously, the system 50 can use dynamic quantization to optimize the models by reducing the precision of their numerical values, particularly weights and activations, which can be performed on-the-fly during model operation. Unlike static quantization, which pre-processes and fixes the bit-width of these values before deployment, dynamic quantization adjusts the precision adaptively as data flows through the model. This ensures an optimal balance between computational efficiency and model accuracy, as the bit precision can be tailored to different parts of the model or varying input data.

Example benefits of dynamic quantization include, by representing values with fewer bits, memory usage is decreased, and computation becomes more efficient; which is especially advantageous for hardware with constrained resources. Additionally, the adaptability of dynamic quantization allows hardware implementations to conserve power, as lower-precision arithmetic requires less energy, resulting in more energy-efficient machine learning inference.

To implement dynamic quantization in hardware, the system 50 can integrate a real-time profiling unit to monitor the range and distribution of activations and weights, adjusting bit-widths accordingly. A configurable quantization unit alters precision levels based on this profiling, leveraging look-up tables or specialized circuits for rapid conversion. Dedicated, precision-specific arithmetic units handle computations efficiently, while dynamic routing ensures data is directed to appropriate units. On-chip memory is managed to cater to varying precision data, and power management techniques exploit reduced power needs during lower precision operations. Throughout dynamic optimization, an integrated feedback mechanism can monitor output quality, adjusting quantization levels to maintain accuracy, while a user interface can provide control over quantization parameters.

The system 50 can also implement dynamic supply voltage scaling (DSVS), which is an approach to dynamically modify voltage requirements in response to computational needs. For the system 50, DSVS markedly conserves power during machine learning model inference by lowering the voltage during cycles with lower computational intensity or when maximum precision isn't crucial. By tailoring the voltage to a present computational load, the system 50 can curtail excessive power usage, thereby extending battery duration. Furthermore, in some cases, the system 50 incorporates an error-detection feature that, upon identifying an error, elevates the voltage and re-executes the computation.

In a particular case, the system 50 can implement DSVS by using a controller governed by a voltage schedule, which can be predetermined from evaluating computational load of the model during emulation. In real-time, should the system 50 identify heightened computational demands or an error (like breaches in setup and hold time), the system 50 can dynamically elevate the supply voltage; otherwise, lowering the voltage to sustain optimal power efficiency.

As referenced herein, the present inventors conducted example experiments to verify the substantial advantages of the present embodiments.

The example experiments used a public database from the of sleep studies to train and test the DL model. The database contained 5 subsets of adult polysomnography recordings, which were labeled by experts. Subsets that were used contained recordings of 19 subjects labeled per 20-second EEG epoch based on the Rechtschaffen and Kales (R&K) standard; and contained recordings of 62 subjects labeled per 30-second EEG epoch based on the American Academy of Sleep Medicine (AASM) standard. All EEG recordings had a sampling rate of 256 Hz.

To evaluate the model performance, a leave-one-subject out and leave-two-subjects-out cross-validation strategies were used. 10% of the test subjects' data were used for fine-tuning per validation, and the remaining 90% of the data were used for testing. All test data were excluded from training. Adam optimizer was used for training with lr=10⁻⁴, beta1=0.9, beta2=0.999 for 100 epochs. L2 weight decay was adopted to prevent overfitting with a value of 10⁻³. A batch size of 256 was used for general training. The sequence length of 3 was used in sequential learning. The extracted features of the previous two segments and the current segment were used as input to the sequential learning. Overall accuracy (ACC), macro F1-score, Cohen's Kappa coefficient (k), and per-class accuracy were used to evaluate the performance of the model.

TABLE 2 summarizes the performance of the system 50 based on the evaluation in the example experiments. Performance before and after 8-bit quantization is shown. The result suggests a marginal degradation of approximately 1% for significant hardware savings. TABLE 3 shows the performance of the system 50 compared to other approaches. On average of the 81 subjects, the accuracy of our model is 85.8% and the F1-score is 79%, which are comparable to other DL architectures that are implemented on much more powerful external computing devices.

TABLE 2

| | Overall Metrics | | | Per-class ACC | | | | |
|---|---|---|---|---|---|---|---|---|
| Quantization | ACC | F1 | k | W | N1 | N2 | N3 | REM |
| Before | 85.5 | 76.6 | 78.6 | 83.2 | 43.5 | 89.3 | 84.2 | 82.7 |
| After | 84.9 | 75.8 | 77.8 | 82.3 | 42.0 | 89.1 | 83.9 | 81.8 |

TABLE 3

| | | | Overall Metrics | | |
|---|---|---|---|---|---|
| Data | Publication | Methods | ACC | F1 | k |
| 30-second | EOGNET | CNN + RNN | 83.1 | 76.4 | 75 |
| subset | IITNet | CNN + RNN | 86.6 | 80.8 | 80 |
| | DeepSleepNet | CNN + RNN | 86.2 | 81.7 | 80 |
| | TinySleepNet | CNN + RNN | 87.5 | 83.2 | 82 |
| | Present Embodiments | CNN + RNN | 86.1 | 80.0 | 79.4 |
| 20-second | MetaSleepLearner | CNN | 77.3 | 69.9 | 68 |
| subset | TinySleepNet | CNN + RNN | 82.6 | 75.5 | 75 |
| | Present Embodiments | CNN + RNN | 84.9 | 75.8 | 77.8 |

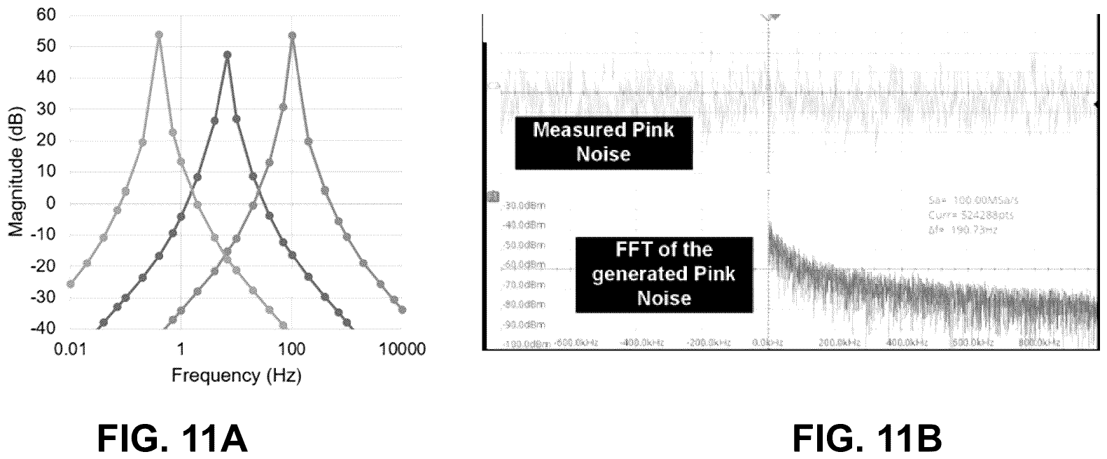

In the example experiments, the analog front-end and auditory stimulation were fully characterized. FIG. 11A shows the experimentally measured frequency response of the biquad filter and FIG. 11B shows experimentally measured time-domain and spectrum of the generated pink noise.

Advantageously, the present embodiments provide an auditory neuromodulation system that implements a low-power DL model and capable of using, for example, FPGA-acceleration to deliver real-time sleep stage classification with high-quality performance. In addition, the present embodiments advantageously implement resource reusing, pipelining, model pruning, and other low-power design techniques to optimize performance.

Although the foregoing has been described with reference to certain specific embodiments, various modifications thereto will be apparent to those skilled in the art without departing from the spirit and scope of the invention as outlined in the appended claims. The entire disclosures of all references recited above are incorporated herein by reference.

1. A low-power neuromodulation system comprising a controller to receive electroencephalogram (EEG) signals from one or more pairs of electrodes, the controller receives power from a power source, the controller comprising a processor, memory, integrated circuitry, field programmable gate array, or a combination thereof, to execute:
an analog front-end (AFE) module to digitize and amplify the received EEG signals;
a processing module to classify sleep stages using a deep learning model, the deep learning model taking the digitized and amplified EEG signals as input, the deep learning model comprising representation learning to capture time-invariant information from the input, sequential learning to capture sleep stage transition using features encoded in the representation learning, and a dense network to generate a prediction for the sleep stages using the captured time-invariant information and the captured sleep stage transitions; and
an output module to output the classification of sleep stages.

2. The low-power neuromodulation system of claim 1, wherein the representation learning comprises one or more Convolutional Neural Network (CNN) paths, each CNN path trained to learn features from the received EEG signals using a distinct time scale.

3. The low-power neuromodulation system of claim 1, wherein the features are in either an analog domain or a digital domain.

4. The low-power neuromodulation system of claim 1, wherein the output of the representation learning and the output of the sequential learning are provided as residual connections to the dense network.

5. The low-power neuromodulation system of claim 1, the system further comprising an auditory stimulator, and wherein the processing module further determines neuromodulation auditory feedback using the classified sleep stages such that the auditory feedback is delivered in phase with sleep oscillation, and wherein the output module further outputs the auditory feedback with the auditory stimulator.

6. The low-power neuromodulation system of claim 5, wherein the auditory feedback comprises in-phase pink noise.

7. The low-power neuromodulation system of claim 5, wherein the processing module filters the received EEG signals to determine occurrence of slow-wave oscillation, and wherein the processing module outputs the auditory feedback during specific phases of the occurrence of the slow-wave oscillation.

8. The low-power neuromodulation system of claim 1, wherein the received EEG signals comprise only one EEG channel.

9. The low-power neuromodulation system of claim 1, wherein the deep learning model uses long kernels using at least one of: memory hierarchies, employing several processing elements (PEs) as part of the processor to manage different segments of the received EEG signals or kernel concurrently, kernel compression, and use of a minimum precision per kernel.

10. The low-power neuromodulation system of claim 1, wherein the processing module performs dynamic supply voltage scaling by instructing adjustment of voltage from the power source based on layers of the deep learning model being processed, instructing lower supply voltage during low-precision layers.

11. A method for low-power neuromodulation, the method comprising:
receiving electroencephalogram (EEG) signals;
digitizing and amplifying the received EEG signals;
classifying sleep stages using a deep learning model, the deep learning model taking the digitized and amplified EEG signals as input, the deep learning model comprising representation learning to capture time-invariant information from the input, sequential learning to capture sleep stage transition using features encoded in the representation learning, and a dense network to generate a prediction for the sleep stages using the captured time-invariant information and the captured sleep stage transitions; and
outputting the classification of sleep stages.

12. The method of claim 11, wherein the representation learning comprises one or more Convolutional Neural Network (CNN) paths, each CNN path trained to learn features from the received EEG signals using a distinct time scale.

13. The method of claim 11, wherein the features are in either an analog domain or a digital domain.

14. The method of claim 11, wherein the output of the representation learning and the output of the sequential learning are provided as residual connections to the dense network.

15. The method of claim 11, the method further comprising determining neuromodulation auditory feedback using the classified sleep stages such that the auditory feedback is delivered in phase with sleep oscillation, and the method further comprising outputting the auditory feedback.

16. The method of claim 15, wherein the auditory feedback comprises in-phase pink noise.

17. The method of claim 15, the method further comprising filtering the received EEG signals to determine occurrence of slow-wave oscillation, and wherein the auditory feedback is outputted during specific phases of the occurrence of the slow-wave oscillation.

18. The method of claim 11, wherein the received EEG signals comprise only one EEG channel.

19. The method of claim 11, wherein the deep learning model uses long kernels using at least one of: memory hierarchies, employing several processing elements (PEs) as part of the processor to manage different segments of the received EEG signals or kernel concurrently, kernel compression, and use of a minimum precision per kernel.

20. The method of claim 11, the method further comprising performing dynamic supply voltage scaling by instructing adjustment of voltage based on layers of the deep learning model being processed, instructing lower supply voltage during low-precision layers.
